(19) United States
(12) Patent Application Publication
(10) Pub. No.: US 2008/0132757 A1
(43) Pub. Date: Jun. 5, 2008

(54) System and Method for Performing Minimally Invasive Surgery Using a Multi-Channel Catheter

(75) Inventor: Nora T. Tgavalekos, Tewksbury, MA (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/566,021

(22) Filed: Dec. 1, 2006

(51) Int. Cl. A61B 1/012

(52) U.S. Cl. 600/104; 600/117; 600/116

(57) ABSTRACT

A system and method from minimally invasive lung surgery employ a bronchoscope with a multi-channel catheter disposed in a channel of the bronchoscope. A transmitting antenna disposed at a distal end of the multi-channel catheter allows the distal end to be tracked in a tracking image during a surgical procedure.

### CROSS REFERENCE TO RELATED APPLICATIONS

Not Applicable.

### STATEMENT REGARDING FEDERALLY SPONSORED RESEARCH

Not Applicable.

### FIELD OF THE INVENTION

This invention relates generally to surgical devices and surgical procedures and, more particularly, to surgical devices and surgical procedures used for minimally invasive surgery.

### BACKGROUND OF THE INVENTION

The lungs are subject to a variety of diseases, including emphysema. Emphysema is a disease in which elasticity of the lung is degraded and alveolar tissue structures are damaged. The diseased tissues can induce collapse of small airways, which results in air being trapped in regions of the lung. The trapped air can result in hyperinflation of the regions of the lung.

As is known, a variety of techniques are used to release the trapped air in the lung regions and to seal the lung region from further hyperinflation. These procedures are often referred to as “lung volume reduction surgery” (LVRS) procedures.

Of those afflicted with emphysema, only about twenty percent are eligible for LVRS, particularly since the lung region hyperinflation often occurs in a late stage of emphysema when a patient tends to be in a clinically fragile state. LVRS is used to remove regions of the lung from physiological operation.

Conventional techniques used to perform LVRS include “median stemotomy” and “video-assisted thoracic” techniques, both of which are invasive techniques. Median sternotomy involves cutting through the sternum to expose the chest cavity. Video-assisted thoracic techniques involves making small incisions in both sides of the chest to allow insertion of surgical instruments having optical viewing capability between the ribs and into the chest cavity.

As is known, a bronchoscope can be inserted into the lung without incision, for example, through the trachea of the patient. The bronchoscope has optical viewing capability, which can be used to optically view internal regions of the lung for diagnostic purposes. However, it is difficult to identify the position of the bronchoscope in the lung, even with the direct optical viewing capability. It will be appreciated that the direct optical imaging provided by the bronchoscope does not provide a reliable positioning of the bronchoscope in relation to anatomical structures in the lung. Essentially, the surgeon can easily become lost as he traverses the lung passageways with the bronchoscope using the bronchoscopic optical image.

As is also known, a variety of other real-time imaging systems, e.g., a computer aided tomography (CT) system, can be used to view internal regions of the lung, or instruments inserted into the lung. Some forms of real-time imaging systems provide a three-dimensional view, while others provide a view in only two dimensions.

As is also known, a catheter can be inserted into the lung. However, the position of the catheter in the lung is generally not well known. A position of the catheter inserted into the lung can also be viewed with some of the real-time imaging systems.

The other real-time imaging systems, e.g., the CT system, though providing, in some modalities, good images of the bronchoscope or catheter relative to lung structures in real time, tend to emit radiation, (e.g., x-rays), harmful to both the patient and to the surgical staff. Furthermore, some real-time imaging systems (e.g., x-ray fluoroscopic system) provide only two-dimensional images against which the position of the bronchoscope or catheter can be viewed, which tends to be insufficient for many lung surgical procedures.

Tracking (or navigation) systems that can track the position of surgical instruments in the body during a medical procedure are known. The tracking systems employ various combinations of transmitting antennas and receiving antennas adapted to transmit and receive electromagnetic energy. Some types of conventional tracking systems are described in U.S. patent application Ser. No. 10/611,112, filed Jul. 1, 2003, entitled “Electromagnetic Tracking System Method Using Single-Coil Transmitter,” U.S. Pat. No. 7,015,859, issued Mar. 21, 2006, entitled “Electromagnetic Tracking System and Method Using a Three-Coil Wireless Transmitter,” U.S. Pat. No. 5,377,678, issued Jan. 3, 1995, entitled “Tracking System to Follow the Position and Orientation of a Device with Radiofrequency Fields,” and U.S. Pat. No. 5,251,635, issued Oct. 12, 1993, entitled “Stereoscopic X-Ray Fluoroscopy System Using Radiofrequency Fields.”

Some tracking systems have been adapted to track flexible probes inserted into the body for minimally invasive surgeries, for example, nasal surgeries. One such system is described in U.S. Pat. No. 6,445,943, issued Sep. 3, 2002, entitled “Position Tracking System for Use in Medical Applications.” Each of the aforementioned patent applications and patents are incorporated by reference herein in the entirety.

None of the above-identified tracking systems have been applied to lung surgery, which requires particular procedures described more fully below.

It would, therefore, be desirable to provide an improved system and a method to perform minimally invasive lung surgery, for example, LVRS.

### SUMMARY OF THE INVENTION

In accordance with the present invention, a method of performing surgery includes acquiring an image of a lung of a patient and advancing a bronchoscope into the lung of the patient. The method also includes inserting a multi-channel catheter into the lung of the patient by way of a channel in the bronchoscope and generating a tracking image showing a representation of the distal end of a multi-channel catheter relative to the image of the lung. The multi-channel catheter has a plurality of channels. While tracking the distal end of the multi-channel catheter in the tracking image, the multi-channel catheter is advanced to a target region of the lung in accordance with the tracking image. While tracking the distal end of the multi-channel catheter in the tracking image and while maintaining the distal end of the multi-channel catheter at the target region of the lung, a corrective medical procedure is performed at the target region of the lung.

In accordance with another aspect of the present invention, apparatus for performing surgery includes a bronchoscope having a channel disposed along a longitudinal dimension of the bronchoscope. The apparatus further includes a multi-channel catheter disposed in the channel and adapted to move in a direction generally parallel to the longitudinal dimension of the bronchoscope. The multi-channel catheter includes at least two channels generally parallel to a longitudinal dimension of and within the multi-channel catheter. The multi-channel catheter also includes a distal end. The apparatus further includes a catheter antenna fixedly coupled to the multi-channel catheter proximate to the distal end of the multi-channel catheter. The catheter antenna is adapted to be tracked during a corrective medical procedure at a target region of the lung in a tracking image showing a representation of the distal end of the multi-channel catheter relative to an image of a lung of a patient.

### BRIEF DESCRIPTION OF THE DRAWINGS

The foregoing features of the invention, as well as the invention itself may be more fully understood from the following detailed description of the drawings, in which:

FIG. 1 is a block diagram showing a system having a bronchoscope and a multi-channel catheter, which can be used for non-invasive lung surgery, including, but not limited to, lung volume reduction surgery (LVRS);

FIG. 2 is a block diagram showing a portion of the bronchoscope and multi-channel catheter of FIG. 1 in greater detail, the multi-channel catheter having a distal end;

FIG. 2A is a block diagram showing an alternate distal end of the multi-channel catheter of FIG. 2;

FIG. 2B is a block diagram showing another alternate distal end of the multi-channel catheter of FIG. 2;

FIG. 3 is a cross section of the multi-channel catheter of FIG. 2; and

FIG. 4 is a flow chart of a process used to perform non-invasive lung surgery using a system as in FIG. 1.

### DETAILED DESCRIPTION OF THE INVENTION

Before describing the present invention, some introductory concepts and terminology are explained. As used herein, the term “lung volume reduction surgery” or “LVRS” is used to describe a surgery used to either remove or to seal off from further physiological function, a portion of a lung.

As used herein, the term “real-time” is used to describe computer operations that are performed without appreciable delay, for example, at the speed of the computer processing, or at the speed of computer communications.

While the system and method are described herein to perform LVRS, it should be understood that the system and methods can be used to perform other non-invasive lung surgeries, including, but not limited to, surgeries that involve thermal ablation techniques, or laser techniques.

As is known, a conventional bronchoscopic system has a conventional bronchoscope adapted to be inserted into the lung. The conventional bronchoscopic system can generally only provide an optical view of internal regions of a lung. To this end, the conventional bronchoscope includes a flexible portion having at least one optical fiber therein, for illumination of and viewing of the internal portion of the lung. However, it should be understood that the bronchoscope described herein is used in conjunction with a multi-channel catheter, as described more fully below.

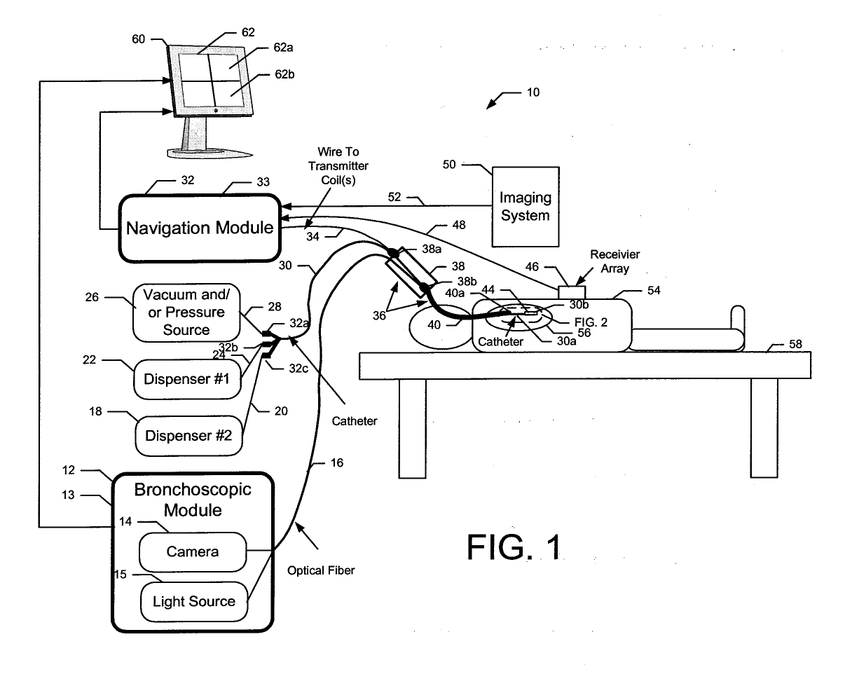

Referring to FIG. 1, an exemplary system 10, that can be used for non-invasive lung surgery, including, but not limited to, lung volume reduction surgery (LVRS), includes a bronchoscopic system 12.

The bronchoscopic system 12 can include a bronchosopic module 13 coupled to a bronchoscope 36 via at least one optical fiber 16. In particular, the bronchoscope 36 can be coupled to a camera 14 and to a light source 15 within the bronchoscopic module 13. The light source 15 can provide illumination at a distal end of the optical fiber. The camera 14 can be a charge coupled device (CCD) camera, adapted to provide an optical image associated with a region proximate to the distal end of the optical fiber 16, which can be displayed on a display device 60.

The bronchoscope 36 can include a body 38 and a flexible portion 40 adapted to be inserted into a lung 56 of a patient 54. It should be understood that the patient 54 is not a part of the system 10, but is shown for clarity. The flexible portion 40 has a distal end 40a, to which the distal end of the optical fiber 16 can extend. Therefore, the distal end 40a is representative of both the distal end of the flexible portion 40 of the bronchoscope 36 and also of the distal end of the optical fiber 16.

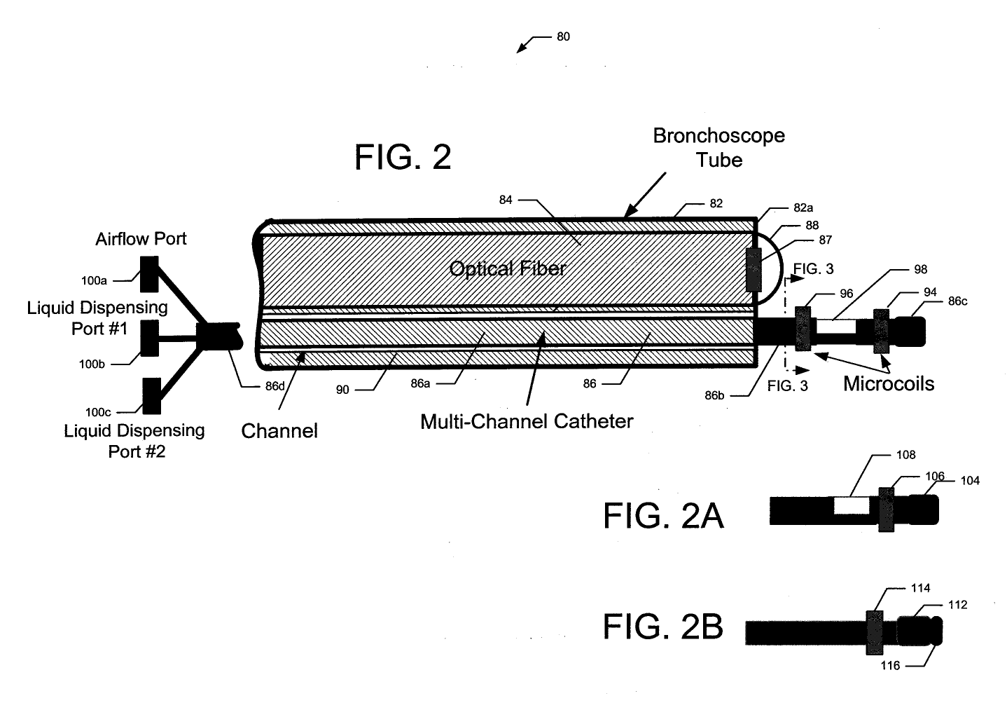

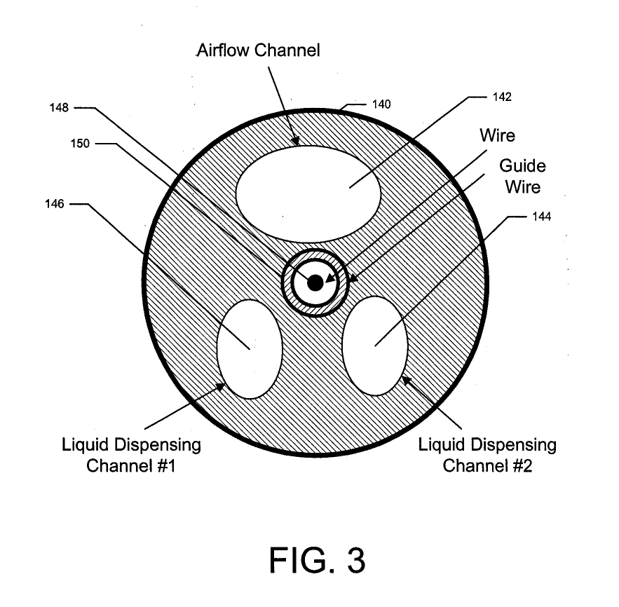

A multi-channel catheter 30 can be disposed in a channel within the bronchoscope 36, both within the body 38 and within the flexible portion 40. The multi-channel catheter 30 can be movable in a direction generally parallel to a longitudinal dimension of the flexible portion 40 of the bronchoscope 36. In some embodiments, the multi-channel catheter 30 can include three channels (not shown) generally parallel to a longitudinal dimension of and within the multi-channel catheter 30. However, in other embodiments, the multi-channel catheter 30 can include more than three or fewer than three channels. The channels are described more fully below in conjunction with FIGS. 2 and 3.

The multi-channel catheter 30 can be movable so as to extend beyond the distal end 40a of the flexible portion 40 of the bronchoscope 36, resulting in an extended portion 30a of the multi-channel catheter 30, which has a distal end 30b. The extend portion 30a of the multi-channel catheter 30 can include a transmitting antenna 44, for example, a microcoil antenna, described more fully below, which is disposed proximate to the distal end 30b of the multi-channel catheter 30. The transmitting antenna 44 can be coupled to one or more wires 34,

The multi-channel catheter 30 can include ports, for example, a first port 32a, a second port 32b and a third port 32c, each port coupled to a channel in the multi-channel catheter 30. The first port 32a can also be coupled, for example, to a vacuum or pressure source 26, adapted to supply gas having a pressure or vacuum to the port 32a. The gas can include, but is not limited to, filtered air, or nitrogen. The second port 32b can be coupled, for example, to a first liquid dispenser 22, adapted to inject a first liquid into the port 32b. The third port 32c can be coupled, for example, to a second liquid dispenser 18, adapted to inject a second liquid into the port 32b.

While the multi-channel catheter 30 is shown and described to have one vacuum and/or pressure port 32a, and two liquid dispensing ports 32b, 32c, in other embodiments, the multi-channel catheter 30 can have more than three of fewer than three ports and a corresponding number of internal channels. Each one of the channels can be either a vacuum and/or pressure port, a liquid dispensing port, or a liquid removal port.

The multi-channel catheter 30 and the microcoil wires 34 merge at a junction 38a, where the wires 34 can become integral to the multi-channel catheter 30. This arrangement is more fully descried below in conjunction with FIG. 3. The junction 38a can be coupled to the body 38 of the bronchoscope 36. In other embodiments, the junction 38a can be separate from the body 38.

The optical fiber 16 and the multi-channel catheter 30 (including the wires 34) merge at a junction 38b. At the junction 38b, the multi-channel catheter 30 and the optical fiber 16 can remain separate but are both disposed with in the flexible portion 40 of the bronchoscope 36. This arrangement is more fully descried below in conjunction with FIG. 2. The junction 38b can be coupled to the body 38 of the bronchoscope 36. In other embodiments, the junction 38b can be separate from the body 38. In some embodiments, the junctions 38a, 38b are the same junction, at which the optical fiber 16, the multi-channel catheter 30, and the wire 34 merge, wherein the wire 34 can become integral to the multi-channel catheter 30, and the optical fiber 16 can remain separate but both disposed within the flexible portion 40 of the bronchoscope 36.

The system 10 can also include a navigation system 32. The navigation system 32, with the exception of modification and adaptations described more fully below, can generally be of a type previously described, for example, in U.S. patent application Ser. No. 10/611,112, filed Jul. 1, 2003, entitled “Electromagnetic Tracking System Method Using Single-Coil Transmitter,” U.S. Pat. No. 7,015,859, issued Mar. 21, 2006, entitled “Electromagnetic Tracking System and Method Using a Three-Coil Wireless Transmitter,” U.S. Pat. No. 5,377,678, issued Jan. 3, 1995, entitled “Tracking System to Follow the Position and Orientation of a Device with Radiofrequency Fields,” U.S. Pat. No. 5,251,635, issued Oct. 12, 1993, entitled “Stereoscopic X-Ray Fluoroscopy System Using Radiofrequency Fields,” Or U.S. Pat. No. 6,445,943, issued Sep. 3, 2002, entitled “Position Tracking System for Use in Medical Applications,” each of which is incorporated by reference herein in its entirety.

The navigation system 32 can include a navigation module 33 coupled to the transmitting antenna 44 with the wires 34. In some embodiments, the wires 34 comprise a miniature coaxial cable. The navigation system 32 can also include a receiving array 46, for example, an array of coil antennas, coupled with one or more wires 48 to the navigation module 33.

The navigation module 33 is coupled to an imaging system 50 with one or more wires 52. The imaging system 50 can include, but is not limited to, a computer-aided tomography (CT) system, a magnetic resonance imaging (MRI) system, an x-ray system, an x-ray fluoroscopy system, and an optical imaging system.

The imaging system 50 provides at least one image of the patient 54 to the navigation module 33. In some embodiments, the imaging system 50 generates the image at a time prior to, or early in, a surgical procedure, described more fully below in conjunction with FIG. 5. In some embodiments, the imaging system 50 can be replaced by a digital storage medium, for example, a hard disk, adapted to store a digital representation of an image of the patient 54. The digital representation of the image can be provided to the navigation module 33.

The navigation module 33 can provide a so-called “tracking image” on the display device 60. In some embodiments, the tracking image and the above-described optical image provided by the camera 14 can be provided as respective separate panes 62 on the display device. For example, the tracking image and the bronchoscopic image can be provided in separate panes 62a, 62b. In other embodiments, the tracking image can be displayed on a different display device (not shown) from the display device 60, on which the optical image is displayed.

In operation, the tracking image generated by the navigation module 33 provides a representation of a position and, in some embodiments, an orientation, of the distal end 30b (proximate to the microcoil 44) of the catheter 30 relative to the image provided by the imaging system 50. While the image provided by the imaging system 50 is generally not provided in real-time, the representation of the distal end 30b relative to the image can be updated in real-time in the tracking image. However, the image provided by the imaging system 50 can also be provided in real-time, of from time to time, during a surgical procedure.

In some embodiments, the distal end 40a of the flexible portion 40 of the bronchoscope 36 also includes a transmitting antenna (not shown) coupled to the navigation module with one or more wires (not shown). In these embodiments, the tracking image can also show a representation of a position and, in some embodiments, an orientation, of the distal end 40a of the flexible portion 40 relative to the image provided by the imaging system 50.

One particular way in which the system 10 can be used is described below in conjunction with FIG. 5. However, let it suffice here to say, that the bronchoscope 36 can be inserted into the lung 56 of the patient 54. The multi-channel catheter 30 having the tracking antenna 44 can be inserted into the lung 56 of the patient 54, via the bronchoscope 36. The distal end 30b of the multi-channel catheter 30 can be moved and also tracked with the navigation system 32, resulting in placement of the distal end 30b of the multi-channel catheter 30 at a desired location (target region) in the lung 56 of the patient 54.

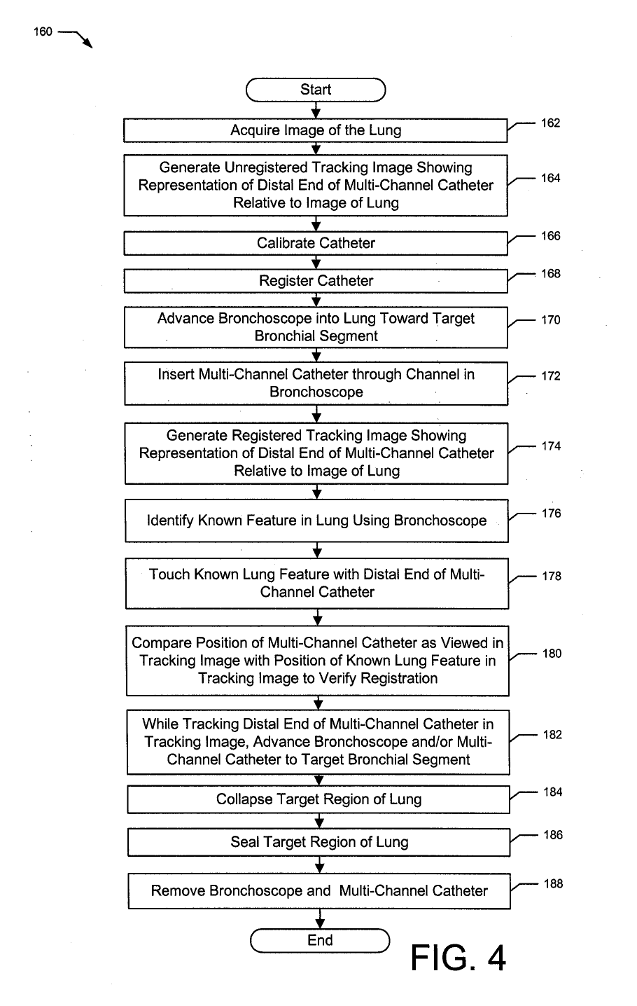

One or more of a variety of surgical procedures can then be performed via the multi-channel catheter once it is at a desired location in the lung 56, while simultaneously tracking the distal end 30b of the multi-channel catheter 30 via the tracking display upon the monitor 60. Exemplary procedures are described below in conjunction with FIG. 4.

Referring now to FIG. 2, a bronchoscope tube 82 can be the same as or similar to part of the flexible portion 40 of the bronchoscope 36 of FIG. 1. An optical fiber 84 is disposed in the bronchoscope tube 84. The optical fiber 84 can be the same as or similar to the optical fiber 16 of FIG. 1. A lens 88 can be coupled to the optical fiber 84. A portion 86a of a multi-channel catheter 86 is disposed in a channel 90 within the bronchoscope tube 82, and an extended portion 86b of the multi-channel catheter 86 can extend by a movable amount beyond the bronchoscope tube 82. The multi-channel catheter 86 can be the same as or similar to the multi-channel catheter 30 of FIG. 1 having the wire 34 of FIG. 1 therein (not shown).

The extended portion 86b of the multi-channel catheter 86 has a distal end 86c. Two microcoil transmitting antennas 94, 96 can be disposed proximate to the distal end 86c. In some embodiments, a surgical device 98 can also be disposed proximate to the distal end 86c. The microcoil antennas 94, 96 can be the same as or similar to the transmitting antenna 44 of FIG. 1.

In some arrangements, each one of the microcoil antennas 94, 96 consists of a single coil, as described, for example, in the above mentioned U.S. patent application Ser. No. 10/611,112, filed Jul. 1, 2003, entitled “Electromagnetic Tracking System Method Using Single-Coil Transmitter.” However, in other embodiments, each one of the microcoil antennas 94, 96 can include a plurality of microcoil antennas, as described, for example, in U.S. Pat. No. 7,015,859, issued Mar. 21, 2006, entitled “Electromagnetic Tracking System and Method Using a Three-Coil Wireless Transmitter.”

In some embodiments, the surgical device 98 comprises an inflatable balloon coupled to one of the channels within the multi-channel catheter 86, for example to an airflow channel described more fully below in conjunction with FIG. 3.

Also shown, another portion 86d of the multi-channel catheter 86 can include three ports, for example an airflow port 100a, a first liquid dispensing port 100b, and a second liquid dispensing port, which can be the same as or similar to the ports 32a, 32b, 32c, respectively, of FIG. 1.

The portion 86a of the multi-channel catheter 86 is adapted to move in the channel 90 in a direction generally parallel to a longitudinal dimension of the bronchoscope tube 82, i.e., to the left to right as shown. Therefore, the distal end 86c of the multi-channel catheter 86 can extend beyond the bronchoscope tube 82 by a controlled amount. In some embodiments, the multi-channel catheter 86 also include a guiding device (not shown), for example, a guide wire, adapted to allow a surgeon to move the distal end 86c of the catheter 86 in direction other than generally parallel to the longitudinal dimension of the bronchoscope tube 82. A guide wire is descried below in conjunction with FIG. 3

As will be apparent from discussion above, a position and, in some embodiments, an orientation of, the microcoil antennas 94, 96 relative to an image (e.g., a CT image of the patient) can be displayed, for example, on the display device 60 of FIG. 1. Therefore, with this arrangement, portion of the multi-channel catheter 86 near the distal end 86c of the catheter 86 can be tracked during a surgical procedure in real-time.

It should be understood that tracking of the distal end 86c of the catheter 86 during the surgical procedure has particular advantages, particularly when the surgical procedure involves a portion of the surgical procedure performed at a first specific location in the body and another portion of the surgical procedure performed at a second specific location. In this case, the distal end 86c of the catheter 86 is first moved to and tracked to the first specific location by a surgeon and is then moved to and tracked to the second specific location by the surgeon. At each one of the first and second locations, a corresponding portion of the surgical procedure can be performed. Exemplary procedures are described below in conjunction with FIG. 4.

In some embodiments, another transmitting antenna 87, for example, another microcoil antenna, can be disposed proximate to a distal end 82a of the bronchoscope tube 82. With this arrangement, it will be understood that a position and, in some embodiments, an orientation of, the microcoil antenna 87 relative to the image (e.g., a CT image of the patient) can also be displayed, for example, on the display device 60 of FIG. 1. Therefore, with this arrangement, both the distal end 82a of the bronchoscope tube, and also the distal end 86c of the catheter can both be tracked during a surgical procedure in real-time.

Referring now to FIG. 2A, an alternate arrangement of a portion of a multi-channel catheter, which can be used in place of the multi-channel catheter 86 of FIG. 2, includes a distal end 104, one microcoil transmitting antenna 106, and a surgical device 108. The surgical device 108 can be the same as or similar to the surgical device 98 of FIG. 2.

It should be understood that, even having one microcoil transmitting antenna 106, the navigation module 33 of FIG. 1 can track a position and an orientation of the transmitting antenna 106.

Referring now to FIG. 2B, another alternate arrangement of a portion of a multi-channel catheter, which can be used in place of the multi-channel catheter 86 of FIG. 2, includes a distal end 112, one microcoil transmitting antenna 114, and a surgical device 116. In some embodiments, the surgical device 116 is a thermal device. In some embodiments, the thermal device can generate heat in response to an electrical current passing though the surgical device. In other embodiments, the thermal device can generate cold in response to an electrical current passing though the surgical device. A Pelletier device is one such device. In some embodiments, the device 116 is a laser.

In still other embodiments, the surgical device 116 is a reservoir coupled to one of the channels within the multi-channel catheter, e.g., 86, of FIG. 1. With these arrangements a hot or a cold liquid, e.g., liquid nitrogen, can be dispensed into the reservoir 116.

Referring now to FIG. 3, a cross section of a multi channel catheter 140 can be representative of a cross section of the portion 86b of the multi-channel catheter 86 of FIG. 2.

The multi-channel catheter 140 includes an airflow channel 142 adapted to provide a passage for a gas, for example, nitrogen, in either direction along a length of the airflow channel 142. The multi-channel catheter 140 also includes a liquid dispensing channel 148 adapted to provide a passage for a liquid in either direction. The multi-channel catheter 140 also includes another liquid dispensing channel 144 adapted to provide a passage for a liquid in either direction. It will be understood that the channels 142, 144, 146 extend from the ports 32a-32c of the multi-channel catheter 30 of FIG. 1 to or near to the distal end 30b (FIG. 1) of the of the multi-channel catheter 30. Therefore, referring briefly to FIG. 1, the vacuum and/or pressure source 16, the liquid dispenser 22, and the liquid dispenser 18 can provide gas pressure and/or liquids near to or at the distal end 30b of the multi-channel catheter 30.

The multi-channel catheter 140 can also include a wire 148, for example, a miniature coaxial cable that can couple to the transmitting antenna 44 of FIG. 1. The wire 140 can be the same as or similar to the wires 34 of FIG. 1. The multi-channel catheter 140 can also include a guide wire 150. Exemplary guide wires are described, for example in U.S. Pat. No. 4,832,047, issued May 23, 1989, entitled “Guide Wire Device,” which patent is incorporated by reference herein in its entirety. Let it suffice here to say that a surgeon or other person can manipulate the guide wire 150 in order to guide the distal end (e.g., 30b of FIG. 1) of the catheter 140 during a surgical procedure. In particular, the distal end 30b can be guided in directions substantially perpendicular to a longitudinal dimension of the catheter 140.

While three ports 142, 144, 146 are shown, in other embodiments, the multi-channel catheter can include more that three or fewer than three channels, including other combinations of liquid and airflow channels.

It should be appreciated that FIG. 4 shows a flowchart corresponding to the below contemplated technique which would be implemented with the system 10 (FIG. 1). Rectangular elements (typified by element 162 in FIG. 4), herein denoted “processing blocks,” represent computer software instructions or groups of instructions.

Alternatively, the processing and decision blocks represent steps performed by functionally equivalent circuits such as a digital signal processor circuit or an application specific integrated circuit (ASIC). The flow diagrams do not depict the syntax of any particular programming language. Rather, the flow diagrams illustrate the functional information one of ordinary skill in the art requires to fabricate circuits or to generate computer software to perform the processing required of the particular apparatus. It should be noted that many routine program elements, such as initialization of loops and variables and the use of temporary variables are not shown. It will be appreciated by those of ordinary skill in the art that unless otherwise indicated herein, the particular sequence of blocks described is illustrative only and can be varied without departing from the spirit of the invention. Thus, unless otherwise stated the blocks described below are unordered meaning that, when possible, the steps can be performed in any convenient or desirable order.

Referring to FIG. 4, an exemplary method 160, begins at block 162, where and image of a lung of a patient is acquired, for example, by the imaging system 50 of FIG. 1. The image can be acquired from one of a variety of imaging systems, including, but not limited to, a computer-aided tomography (CT) system, a magnetic resonance imaging (MRI) system, an x-ray system, an x-ray fluoroscopy system, and an optical imaging system.

At block 164, an unregistered tracking image is generated, for example, by the navigation system 32 of FIG. 1. The unregistered tracking image provides a coarse representation of a position, and in some embodiments, an orientation, of a distal end (e.g., 30b, FIG. 1) of a multi-channel catheter (e.g., 30, FIG. 1) relative to the image acquired at block 162. The representation of the position and/or orientation is made more accurate at processing blocks described below.

At block 166, the position and/or orientation of the distal end of the catheter are calibrated at block 166 and registered at block 168. Calibration and registration of a tracking image are known. In general, calibration is a process by which an undistorted coordinate system is established for the position and/or orientation of the distal end of the catheter. Registration is a process by which the undistorted coordinate system is aligned with and matched to a coordinate system of the image acquired at block 162. Having been calibrated at block 166 and registered at 168, the position and/or orientation of the distal end of the catheter can be viewed in subsequent blocks in a registered “tracking image” that provides an accurate representation of a position, and in some embodiments, an orientation, of the distal end (e.g., 30b, FIG. 1) of the multi-channel catheter (e.g., 30, FIG. 1) relative to the image acquired at block 162.

At block 170, a bronchoscope, for example, the bronchoscope 36 of FIG. 1, can be advanced into a lung of a patient. At block 172, the multi-channel catheter, for example, the multi-channel catheter 30 of FIG. 1, is advanced into the lung of the patient via a channel, e.g., the channel 90 of FIG. 2 in the bronchoscope. As described above, the multi-channel catheter includes a transmitting antenna, e.g., 44 of FIG. 1.

At block 174, a registered tracking image is generated and observed as the multi-channel catheter is advanced through the bronchoscope.

At block 176, using the optical imaging provided by the bronchoscope, a known feature can be identified within the lung of the patient. At block 176, the known feature can be touched with the distal end of the multi-channel catheter.

At block 180, a position of the multi-channel catheter as viewed in the registered tracking image is compared with a position of the known feature in the image acquired at block 162. If the match is sufficient, then the process continues to block 182. However, if the match is not sufficient, then further calibration and or registration can be performed, for example, by withdrawing the multi-channel catheter and repeating the processes of blocks 166 and/or 168.

At block 182, while tracking the distal end of the multi-channel catheter in the registered tracking image, either the bronchoscope or the multi-channel catheter or both are advanced and guided (e.g., via the guide wire 150 of FIG. 3) further into the lung, toward a target bronchial segment (i.e., target region). Once the distal end of the multi-channel catheter is at the target region of the lung, at block 184, while still tracking the distal end of the multi-channel catheter in real-time, one or more surgical procedures can be performed.

If the surgery is for lung volume reduction, at block 184, the target region of the lung can be collapsed and at block 186, the target region of the lung can be sealed, all the while tracking the distal end of the multi-channel catheter in the registered tracking image in real-time. In this way, the distal end of the multi-channel catheter can be repositioned during the surgical procedure, for example, in order to collapse and seal more than one region of the lung.

At block 186, the bronchoscope and multi-channel catheter are removed from the lung.

The collapse of the target region o the lung at block 184 and the sealing at block 186 can be performed in a variety of ways. For example, in order to collapse the target region of the lung at block 184, at the desired location (target region) in the lung, a balloon (e.g., 98 of FIG. 2) proximate to the distal end 86c (FIG. 2) of the multi-channel catheter 86 (FIG. 2), can be inflated, for example, via the airflow channel 142 of FIG. 3, blocking a region of the lung. A first liquid, for example an anti-surfactant liquid, can be dispensed into the lung from a first liquid dispenser (e.g., 22, FIG. 1) via a first liquid dispensing channel (e.g., 146, FIG. 3) of the multi-channel catheter, resulting in closure of the region of the lung. In accordance with the sealing of block 186, a second liquid, for example, a fibrin glue, can be can be dispensed into the lung from a second liquid dispenser (e.g., 18, FIG. 1) via a second liquid dispensing channel (e.g., 144, FIG. 3) of the multi-channel catheter, resulting in permanent sealing of the lung region from further entry of air.

In another procedures, in order to collapse the target region of the lung at block 184, at the desired location in the lung, a negative pressure from a vacuum source (e.g., 26, FIG. 1) can be generated in the lung via an airflow channel (e.g., 142, FIG. 3), collapsing the target region of the lung. In accordance with the sealing of block 186, a liquid, for example, a fibrin glue, can be can be dispensed into the lung from a liquid dispenser (e.g., 18, FIG. 1) via a liquid dispensing channel (e.g., 146, FIG. 3) of the multi-channel catheter, resulting in permanent sealing of the lung region from further entry of air. In this procedure, no balloon is used.

In yet another procedure, in order to collapse the target region of the lung at block 186, at the desired location in the lung, a first liquid, for example, an anti surfactant fluid, can be dispensed into the lung from a first liquid dispenser (e.g., 22, FIG. 1) via as first liquid dispensing channel (e.g., 146, FIG. 3) of the multi-channel catheter, resulting in closure of the region of the lung. In accordance with the sealing of block 186, a second liquid, for example, a fibrin glue, can be can be dispensed into the lung from a second liquid dispenser (e.g., 18 FIG. 1) via a second liquid dispensing channel (e.g., 144, FIG. 3) of the multi-channel catheter, resulting in permanent sealing of the lung region from further entry of air. In this procedure, no balloon is used.

In yet another procedure, in order to collapse the target region of the lung at block 186, at the desired location in the lung, a negative pressure from a vacuum source (e.g., 26, FIG. 1) can be generated in the lung via an airflow channel (e.g., 142, FIG. 3), collapsing a region of the lung. In accordance with the sealing of block 186, a high temperature can be generated at the distal end of the multi-channel catheter with a surgical device (e.g., 116, FIG. 2B.) The high temperature can fuse lung tissue together.

In yet another procedure, in order to advance the multi-channel catheter to the target region of the lung, the target region of the lung can be expanded instead of collapsed by a positive pressure from a pressure source (e.g., 26, FIG. 1) via an airflow channel (e.g., 142, FIG. 3). In order to remove a growth from the lung, a freezing temperature can be generated at the distal end of the multi-channel catheter with a surgical device (e.g., 1116, FIG. 2B). The freezing temperature can result in death and absorption of the frozen lung tissue.

The surgical procedures described above are not intended to limit the scope of the invention to only those procedures.

All references cited herein are hereby incorporated herein by reference in their entirety.

Having described preferred embodiments of the invention, it will now become apparent to one of ordinary skill in the art that other embodiments incorporating their concepts may be used. It is felt therefore that these embodiments should not be limited to disclosed embodiments, but rather should be limited only by the spirit and scope of the appended claims.

What is claimed is:

1. A method of performing surgery, comprising:
acquiring an image of a lung of a patient;
advancing a bronchoscope into the lung of the patient;
inserting a multi-channel catheter into the lung of the patient by way of a channel in the bronchoscope;
generating a tracking image showing a representation of the distal end of a multi-channel catheter relative to the image of the lung, wherein the multi-channel catheter has a plurality of channels;
while tracking the distal end of the multi-channel catheter in the tracking image, advancing the multi-channel catheter to a target region of the lung in accordance with the tracking image; and
while tracking the distal end of the multi-channel catheter in the tracking image and while maintaining the distal end of the multi-channel catheter at the target region of the lung, performing a corrective medical procedure at the target region of the lung.

2. The method of claim 1, wherein the image of the lung is acquired at a time different than and before a time when the tracking image is generated.

3. The method of claim 2, further comprising:
identifying a known anatomical feature internal of the lung of the patient by way of an optical image provided by the bronchoscope;
touching the distal end of the multi-channel catheter to the known feature; and
while tracking the distal end of the multi-channel catheter in the tracking image, comparing the position of the representation of the distal end of the multi-channel catheter in the tracking image with the position of the known anatomical feature in the tracking image.

4. The method of claim 2, wherein the performing the medical procedure comprises:
collapsing a target region of the lung via the multi-channel catheter; and
sealing the target region of the lung via the multi-channel catheter.

5. The method of claim 2, wherein the performing the medical procedure comprises heating the target region of the lung.

6. The method of claim 2, wherein the performing the medical procedure comprises freezing the target region of the lung.

7. The method of claim 2, wherein the multi-channel catheter includes at least two channels generally parallel to a longitudinal dimension of and within the multi-channel catheter.

8. The method of claim 2, wherein the multi-channel catheter includes:
at least one pneumatic channel generally parallel to a longitudinal dimension of and within the multi-channel catheter, wherein the at least one pneumatic channel is adapted to provide a positive or a negative gas pressure proximate to the distal end of the multi-channel catheter; and
at least one liquid channel generally parallel to the longitudinal dimension of and within the multi-channel catheter, wherein the at least one liquid channel is adapted to transfer a liquid to or from a position proximate to the distal end of the multi-channel catheter.

9. The method of claim 2, wherein the multi-channel catheter comprises:
at least two channels generally parallel to a longitudinal dimension of and within the multi-channel catheter, and
an inflatable balloon disposed proximate to the distal end of the multi-channel catheter and pneumatically coupled to at least one of the two channels.

10. The method of claim 2, wherein the multi-channel catheter comprises:
at least two channels generally parallel to a longitudinal dimension of and within the multi-channel catheter, and
a thermal device coupled to the distal end of the multi-channel catheter.

12. The method of claim 2, further including:
applying a positive gas pressure to a balloon disposed proximate to the distal end of the multi-channel catheter via one of the plurality of channels of the multi-channel catheter,
applying a negative gas pressure to the target region of the lung via another one of the plurality of channels of the multi-channel catheter; and
applying a liquid glue to the target region of the lung via another one of the plurality of channels of the multi-channel catheter, wherein the glue is operable to seal the target region of the lung.

13. The method of claim 2, further including:
applying a negative gas pressure to the target region of the lung via one of the plurality of channels of the multi-channel catheter, wherein the negative gas pressure is sufficient to collapse the target region of the lung; and
applying a liquid glue to the target region of the lung via another one of the plurality of channels of the multi-channel catheter, wherein the glue is operable to seal the target region of the lung.

14. The method of claim 2, further including:
applying a constricting liquid to the target region of the via one of the plurality of channels of the multi-channel catheter, wherein the constricting liquid is adapted to collapse the target region of the lung; and
applying a liquid glue to the target region of the lung via another one of the plurality of channels of the multi-channel catheter, wherein the glue is operable to seal the target region of the lung.

15. The method of claim 2, further including:
applying a negative gas pressure to the target region of the lung via one of the plurality of channels of the multi-channel catheter, wherein the negative gas pressure is sufficient to collapse the target region of the lung; and
applying a predetermined temperature to the target region of the lung via the multi-channel catheter.

16. Apparatus for performing surgery, comprising:
a bronchoscope having a channel disposed along a longitudinal dimension of the bronchoscope;
a multi-channel catheter disposed in the channel and adapted to move in a direction generally parallel to the longitudinal dimension of the bronchoscope, wherein the multi-channel catheter comprises at least two channels generally parallel to a longitudinal dimension of and within the multi-channel catheter, and wherein the multi-channel catheter includes a distal end; and
a catheter antenna fixedly coupled to the multi-channel catheter proximate to the distal end of the multi-channel catheter, wherein the catheter antenna is adapted to be tracked during a corrective medical procedure at a target region of the lung in a tracking image showing a representation of the distal end of the multi-channel catheter relative to an image of a lung of a patient.

17. The apparatus of claim 16, wherein the catheter antenna is a coil antenna having a central axis substantially aligned with the longitudinal dimension of the multi-channel catheter.

18. The apparatus of claim 16, wherein the image of the lung is acquired at a time different than and before a time when the tracking image is generated.

19. The apparatus of claim 18, further comprising:
a tracking system adapted to generate the tracking image, wherein the tracking system comprises:
an external antenna disposed external to the patient, wherein the external antenna is adapted to receive electromagnetic energy from or transmit electromagnetic energy to the catheter antenna and to convert the electromagnetic energy received therefrom into the tracking image.

20. The apparatus of claim 18, wherein the multi-channel catheter includes:
at least one pneumatic channel disposed longitudinally within the multi-channel catheter and adapted to provide a positive or a negative gas pressure proximate to the distal end of the multi-channel catheter, and
at least one liquid channel disposed longitudinally within the multi-channel catheter and adapted to transfer a liquid to or from a position proximate to the distal end of the multi-channel catheter.

21. The apparatus of claim 18, wherein the multi-channel catheter further comprises an inflatable balloon fixedly coupled proximate to the distal end of the multi-channel catheter and pneumatically coupled to at least one of the two channels.

22. The apparatus of claim 18, wherein the multi-channel catheter comprises:
a thermal device fixedly coupled proximate to the distal end of the catheter; and
a wire coupled to the thermal device, wherein the thermal device is adapted to generate heat in response to electricity in the wire.

23. The apparatus of claim 18, wherein the multi-channel catheter comprises:
a thermal device fixedly coupled proximate to the distal end of the catheter; and
a wire coupled to the thermal device, wherein the thermal device is adapted to generate cold in response to electricity in the wire.

24. The apparatus fop claim 23, wherein the thermal device comprises a Pelletier device.

25. The apparatus of claim 18, wherein the multi-channel catheter comprises a guide wire operable to guide the distal end of the multi-channel catheter during the surgical procedure.
